(19) United States
(12) Patent Application Publication
(10) Pub. No.: US 2023/0303956 A1
(43) Pub. Date: Sep. 28, 2023

(54) PROCESS SYSTEM FOR BIOREACTOR-BASED CLEAN MEAT PRODUCTION

(71) Applicant: Merck Patent GmbH, Darmstadt (DE)

(72) Inventors: Jean-Louis Weissenbach, Strasbourg (FR); Ryan Sylvia, Tewksbury, MA (US); Almut Von der Brelie, Frankfurt am Main (DE); Melanie Brandl, Ober-ramstadt (DE); Michaela Fesenfeld, Darmstadt (DE)

(21) Appl. No.: 18/042,040

(22) PCT Filed: Aug. 19, 2021
(86) PCT No.: PCT/EP2021/073079

§ 371 Date: Feb. 17, 2023

Related U.S. Application Data
(60) Provisional application No. 63/068,406, filed on Aug. 21, 2020.

(51) Int. Cl. C12M 3/00; C12M 1/00; C12M 1/12; A23L 13/00

(52) U.S. Cl. CPC C12M 21/08; C12M 23/58; C12M 23/40; C12M 25/10; A23L 13/00

(57) ABSTRACT

The present invention is for a closed environment process for the growth and differentiation of cells and the culturing of cells to confluency for the production of tissue. The tissue may be a clean meat product.

### BACKGROUND

Efficient, closed continuous, semi-continuous or batch cell and tissue culture systems are needed, for example, for the production of cells, clean meat or other tissues. Current systems are cumbersome to use, expensive to operate and/or not suitable for scale-up to provide for industrial-scale operation. One such example of a prior art device is provided for in U.S. Pat. No. 8,492,140 (the '140 patent). The device of the '140 patent is a bench top lab-scale device designed specifically for the generation of autologous patient tissue transplants and is not suitable for industry-scale generation of product nor suitable for scale-up to an industry scale device. Further, it does not provide flexibility for alternative culture protocols used during a production cycle necessary for the large-scale production of, for example, clean meat.

Another such example of a prior art device and system is provided for in WO2020/222239 to Aleph Farms, Ltd. (the '239 application) The '239 application discloses a cultivation system for a structured meat product but said system is limited to the utilization of culture bags suspended in a bioreactor and in which cells are grown on a scaffold. Further, the system of the '239 application is directed toward a complicated system necessitating individual peristaltic pumps for each reactor and culture bioreactors that further require to be rotated on their axis to direct fluid flow in the reverse direction.

What is needed in the art are process systems designed for the production of clean meat products, wherein the systems are simple to setup, scalable and flexible to enable the cost effective generation of clean meat.

### SUMMARY OF THE INVENTION

The present invention solves this need by providing a closed, continuous, semi-continuous or batch culture system for cell growth and differentiation followed by tissue growth for the production of, for example, clean meat. The process and system of the present invention solves this problem by utilizing only one bioreactor for cell growth and expansion in a perfusion-type cycle and media exchange to first grow and then expand the cells. Once the cells are grown and expanded, the bioreactor is then used as a media supply vessel. The cells, after being removed from the bioreactor and, optionally, separated from the media by a cell-media separation device, are grown to confluency to form a tissue in a cell differentiation and tissue formation device comprising, preferably, a scaffolding suitable for cell adherence.

Thus, the present invention provides for one or more of the following benefits over the prior art: reduced capital expenditure, single use, extended use, ease of harvest, provides for a closed process (and concurrent reduced chance of contamination), no physical cell transfer is needed outside of the closed system, it is easily scalable (up to 10,000 liters or more), and utilizes a single bioreactor for multiple functions. The bioreactor may be a stirred cell-type bioreactor.

In one aspect, the present invention is a closed environment process for the culture of cells to confluency and form tissue, the process comprising: providing, a system comprising: a cell growth and expansion reactor; one or more tissue formation reactors; and, optionally, a cell retention device; seeding the cell growth and expansion reactor and expanding the cell density within the cell growth and expansion reactor to a desired cell density; once a desired cell density is obtained, optionally processing the cells through the cell retention device thereby transferring the cells to the one or more tissue formation reactors and removing the growth media; and, converting the bioreactor to a media reservoir (e.g., a differentiation media reservoir or cell growth media reservoir) for feeding the tissue growing reactors, and; differentiating and growing said cells in said one or more tissue formation reactors until a desired level of confluency is reached and tissue formed, and harvesting the tissue from said one or more tissue formation reactors.

In another aspect of the present invention the process system is semi-continuous or continuous.

In another aspect of the present invention, the size of the cell growth and expansion reactor is from 0.5 liter to 10,000 liters and 20,000 liters.

In another aspect of the present invention, the size of said cell growth and expansion reactor is from 0.5 liters to 2000 liters.

In another aspect of the present invention, the process further comprises a manifold system to integrate said tissue formation reactors if said process system has two or more of said tissue formation reactors.

In another aspect of the present invention, the process further comprises one or more monitoring systems for i) dissolved oxygen, ii) pH, iii) carbon dioxide, iv) cell waste, v) one or more cell metabolites, vi) temperature, vii) flow rate, viii) cell density and ix) cell viability.

In another aspect of the present invention, the process further comprises bypassing the cell retention device.

In another aspect of the present invention, the process further comprises that the one or more tissue formation reactors are hollow fiber reactors.

In another aspect of the present invention, tissue can be harvested (sterilely or cleanly) from one or more of the one or more tissue formation reactors while maintaining the sterility of the remainder of the system.

In another aspect, a harvested tissue formation reactor can be sterilized and reseeded without compromising the integrity of the reminder of the system.

In another aspect of the present invention, the cells in the bioreactor are adapted to suspension growth, aggregate growth or microcarrier growth.

In another aspect of the present invention, the one or more tissue formation reactors comprise scaffolding for cell attachment.

In another aspect, the present invention comprises a closed environment process for the culture of cells to confluency and form tissue, the process comprising: a) providing: i) a cell growth and expansion reactor, ii) one or more tissue formation reactors; iii) a cell retention device; b) i) seeding said cell growth and expansion reactor and expanding the cell density within the cell growth and expansion reactor, ii) once a desired cell density is obtained, iii) processing the cells through the cell retention device thereby transferring a portion of the cells to the one or more tissue formation reactors and returning a portion of the cells to the bioreactor; and, iv) continuing to transfer cells from the cell growth and expansion reactor as adequate cell density become available in the cell growth and expansion reactor; and, c) i) differentiating and growing said cells in said one or more tissue formation reactors until a desired level of confluency is reached and tissue formed, and ii) harvesting the tissue from said one or more tissue formation reactors.

In another aspect of the present invention, the process further comprises a first reservoir holding cell growth media and a second reservoir holding differentiation media, said cell growth media being delivered to the cell growth and expansion reactor and said differentiation media being delivered to the one or more tissue formation reactors after transferring said cells to the one or more tissue formation reactors.

In another aspect of the present invention, the process system is semi-continuous or continuous.

In another aspect of the present invention, the size of said cell growth and expansion reactor is from 0.5 liters to 20,000 liters.

In another aspect of the present invention, the size of said cell growth and expansion reactor is from 0.5 liter to 2000 liters.

In another aspect of the present invention, the process further comprises a manifold system to integrate said tissue formation reactors if said process system has two or more of said tissue formation reactors.

In another aspect of the present invention, the process further comprises one or more monitoring systems for i) dissolved oxygen, ii) pH, iii) carbon dioxide, iv) cell waste, v) one or more cell metabolites, vi) temperature, vii) flow rate, viii) cell density and ix) cell viability.

In another aspect of the present invention, the process further comprises, wherein the cell retention device can be bypassed.

In another aspect of the present invention, wherein the one or more tissue formation reactors are hollow fiber reactors.

In another aspect of the present invention, tissue can be sterilely harvested from one or more of the one or more tissue formation reactors while maintaining the sterility of the remainder of the system.

In another aspect, a harvested tissue formation reactor can be sterilized and reseeded without compromising the integrity of the reminder of the system.

In another aspect of the present invention, the cells in the bioreactor are adapted to suspension growth, aggregate growth or microcarrier growth.

In another aspect of the present invention, the one or more tissue formation reactors comprise scaffolding for cell attachment.

In another aspect of the present invention, further comprising a separate reservoir for differentiation media that is fluidly connected to said tissue formation reactors.

### FIGURES

FIG. 1 shows an embodiment of the present invention. 1 is growth media comprising cells. 2 is the bioreactor (i.e., growth and expansion reactor). 3 are optional culture parameter sampling devices. 4 is the optional cell retaining device. 5 is a manifold for selectively diverting media and cells between tissue formation reactors. 6 are three tissue formation reactors. 7 are the media input lines for directing the media to an end of the issue formation reactor. 8 are cell seeding lines. 9 is the outflow line from the tissue reactor center tube. 10 is the outflow line from the cell culture chamber of the tissue formation reactor. 12 is the bioreactor/media tank impeller. Not shown is one or more waste lines for removing spent media. Waste lines can be located anywhere between the outflow lines and the bioreactor.

FIG. 2 shows the closed, continuous or semi-continuous culture system of the present invention during the cell proliferation (cell growth and expansion) step of the process of the present invention. No cells or media are being directed toward the tissue formation reactors. Cells are growing and expanding in the bioreactor. FIGS. 2-5 also show a different embodiment of the impeller 12 in tank 2.

FIG. 3 shows the closed, continuous or semi-continuous culture system of the present invention during the differentiation phase of the process of the present invention. Media type is changed from growth media to differentiation media. In other embodiments, the cells may be differentiated partly or totally in the tissue formation reactors.

FIG. 4 shows the closed, continuous or semi-continuous culture system of the present invention during the loading step of the process of the present invention where the tissue formation bioreactors are seeded with cells from the bioreactor.

FIG. 5 shows the closed, continuous or semi-continuous culture system of the present invention during growth (growing) or tissue generation phase of the process to produce the desired tissue. In some embodiments, the cells may differentiate in or continue to differentiate in the tissue formation reactors. In other embodiments the cells are completely differentiated in the bioreactor when loaded into the tissue formation reactors.

FIG. 6 shows a schematic diagram of a prior art process system utilizing a consecutive seed train reactors to increase the cell mass prior to seeding a stirred batch reactor used as the production vessel.

FIG. 7 shows a schematic diagram of the process system of the present invention wherein the cell growth reactor (bioreactor: 2) is used as a media reservoir after cells are seeded into the tissue formation reactors 6. In this aspect of the invention, the tissued formation reactors also function as the differentiation reactors with the addition of differentiation factors 15 to the cells in the tissue formation reactors.

FIG. 8 shows a schematic diagram of the process system of the present invention wherein the cell growth reactor (bioreactor: 2) is used to produce multiple batches of cells (i.e., 2 or more batches of cells) for seeding multiple (i.e., two or more) tissue formation reactors in sequence. After one tissue formation reactor train is harvested (three tissue formation reactor trains are shown in the figure) while maintaining sterile integrity of the remainder of the system, the harvested reactors can be sterilized and reseeded with cells from the bioreactor. In this system, a separate media reservoir is used to feed the tissue formation reactors. 14 is a media storage tank for feeding the tissue formation reactors.

### DETAILED DESCRIPTION ON THE INVENTION

The present invention is directed toward a closed environment process for the culture of cells to confluency and to form tissue. In one embodiment, it is contemplated that the process comprises one or more cell growth and expansion reactors, one or more tissue formation reactors and, optionally, one or more cell retention devices.

In the present invention, a “cell growth and expansion reactor” is defined as a bioreactor suitable for the seeding of a cell type or cell types and maintaining and adjusting culture conditions to achieve the desired rate of growth and expansion of cells to a desired density or confluency. “Maintaining and adjusting” culture conditions is defined herein as meaning regulating a physical parameter necessary for desired cell growth to a set value or value range and, if necessary, adjusting parameters to achieve or maintain a desired rate of cell growth. Such parameters may be one or more of, for example but not limited to, temperature, dissolved gas level (e.g., oxygen and/or carbon dioxide), pH, cell waste (e.g., lactic acid), one or more cell metabolites, flow rate, cell density and cell viability. It is contemplated that the cell growth and expansion reactor is adapted to suspension growth, aggregate growth or microcarrier growth. Cells may be partially or completely differentiated in the cell growth and expansion reactor,

Further, in the present invention a “tissue formation reactor” is defined as a bioreactor specifically designed to permit and enhance the formation of the desired tissue and, in some aspects, the differentiation of cells, preferably the cells grown and expanded in the “cell growth and expansion reactor” of the present invention, to a density reminiscent of natural tissue. Such reactors may consist of an external tube with a top and bottom end cap. The end caps and the tube would have different inlet and outlets in order to allow circulation of the cells and medium in a forward flow as well as in the reverse direction. A smaller tube with a defined porosity is fixed in between the top and bottom endcap inside the external tube. Fluid circulation is allowed through this center tube in both ways. The material used for the assembly of the device could a grade of plastic (food grade, pharma grade), metal (e.g., stainless steel) or alternate material that is known to one of ordinary skill in the art and is in compliance with food industry standards.

The tissue formation reactor may further comprise scaffolding suitable for cell attachment and/or growth. Such scaffolding is known to one of skill in the art and includes hollow fibers, three-dimensional lattices, woven or non-woven materials, etc.

Further still, in the present invention a “cell retention device” is a device or system such as a filtering system specifically designed or adapted to permit the separation of cells (for example, cells grown and expanded in the “cell growth and expansion device” of the present invention) from a liquid (e.g., culture media) in which cells are grown and expanded, or other liquid (e.g., cell compatible saline or buffer) in which the cells have been placed. The cell retention device filters the cells from the media or other liquid. One purpose of this is to eliminate (i.e., remove the cells from) “spent” media. The cells are then resuspended in fresh media. Another purpose is to change one type of media to another media. This may be necessary as the cells grow and expand, a denser culture requiring different media constituents and/or different concentrations of media constituents. Yet another purpose is to concentrate the cells to a higher concentration for effective seeding into, for example, the “tissue formation reactor” of the present invention. And still yet another purpose of the cell retention device is to separate cells from cell clusters or aggregates. The cell retention device of the present invention may perform any or all of these functions singly or simultaneously. The cell retention device of the present invention may perform these functions continuously or intermittently and/or on some or all of the cells from the cell growth and expansion device. The cell retention device may be used during certain steps (but not all steps) in the growth and differentiation of the cells and generation of tissue. For example, the cell retention device may be used to remove cell aggregates before seeding into the tissue formation reactor but not when the cells will be returned to the cell growth and expansion reactor (e.g., during media exchange in the cell growth and expansion reactor). The “cell retention device” of the present invention can be a stand-alone device fluidly connected with the cell growth and expansion device or may be integral with the “cell culture and expansion device” and/or the “tissue formation reactor.” In one embodiment, the cell retention device comprises one or more tangential flow filters (TFF) or single pass tangential flow filters (SPTFF) or other filtering or screening mechanism.

The present invention also contemplates a process for growing, expanding and differentiating cells to form tissue using one or more of the cell growth and expansion reactors, one or more of the tissue formation reactors and, optionally, the cell retention device of the present invention. The process of the present invention, in one embodiment, comprises seeding said cell growth and expansion reactor and expanding the cell density within the cell growth and expansion reactor to a desired cell density, once a desired cell density is obtained; optionally processing the cells through the cell retention device and; then transferring the cells to the one or more tissue formation reactors; removing the growth media from and converting the bioreactor to a differentiation media reservoir for feeding the tissue growing reactor(s); differentiating, if necessary, and growing said cells in said one or more tissue formation reactors until a desired level of confluency is reached and tissue formed and; harvesting the tissue from said one or more tissue formation reactors.

“Seeding” of a bioreactor is defined herein as inoculating a bioreactor with a low density of cells (for example, 1×10^{4/}ml to 1×10^{8/}ml). Once in the bioreactor, the cells reproduce and the population expands/increases. Thus, cell “expansion” is defined herein as increasing the total number of cells per unit volume (typically cells per milliliter (ml)) until a desired cell density is achieved.

The “desired cell density” varies depending on the cell type being cultured (some cell types do not grow to as high a density as others) and end use of the cells. One of ordinary skill in the art, with the teachings of this specification, will be able to determine a desired cell density for a particular purpose.

In some embodiments, the cells may be grown to confluency. With attachment dependent cells (including cells grown on microcarriers) “confluency” is defined herein as covering at least 80%, 85%, 90%, 95%, 98%, 99% or 100% of the available surface area. For suspension cells, confluency is not as well defined in the art but herein is defined as approximately 1×10⁹-1×10¹² cells per ml.

“Cell differentiation/cellular differentiation” is defined herein as the process in which a cell changes from one cell type to another. Usually, the cell changes to a more specialized type. For example, during development of an organism, stem cells differentiate into the specialized cell type that make up the organism. Induced pluripotent stem cells (iPSCs) are a type of stem cell that can be generated directly from a somatic cell. iPSC technology was pioneered by Shinya Yamanaka's lab in Kyoto, Japan, who showed in 2006 that the introduction of four specific genes (Myc, Oct3/4, Sox2 and Klf4) encoding transcription factors could convert somatic cells into pluripotent stem cells. (Takahashi K., Yamanaka S., August 2006, “Induction of pluripotent stem cells from mouse embryonic and adult fibroblast cultures by defined factors,” Cell, 126 (4): 663-676).

Stem cells and iPSCs are capable of differentiating into specialized cells (muscle, nerve, fat, epithelial, etc.) by exposure of the cells to specific differentiating factors. Stem cells and induced pluripotent stem cells can be induced to differentiate into specific desired cell types or cells having the characteristics of specific desired cell types. Characteristics of a specific cell type means that the cells display, for example, morphologies and molecular markers (e.g., cell surface or cytoplasmic markers) unique or indicative of a specific cell type. For example, cells having characteristics of myocytes may exhibit one or more molecular markers such as: PAX7, MYF5, MYOD1 and MYOG (see, for example: M. Shelton et al.,

Methods 101 (2016) 73-84). Cells having characteristics of adipocytes may exhibit one or more of molecular markers such as BMP4, Hox8, Hoxc9, Hoxc5 in white adipocyte progenitors and PRDM16, Diol and Pax3 in brown adipocyte progenitors (see, for example; Mohsen-Kanson, et al., Stem Cells. 2014 June; 32(6):1459-67). It is known in the art what morphological and physiological markers and characteristics can be used to identify a particular cell type or are associated with a particular cell type. Myocytes have been generated from iPSCs by those of skill in the art. See, for example: M. Shelton et al., Methods 101 (2016) 73-84; Laine et al. Skeletal Muscle (2018) 8:1, both of which are incorporated herein in their entirety. Adipocytes have been generated from iPSCs by exposure to, for example, Oct4, Sox2, Klf4 (see, for example: Mohsen-Kanson, et al., Stem Cells. 2014 June; 32(6):1459-67, which is incorporated herein in its entirety). Morphological characteristics of myocytes, adipocytes and other cells/tissues are well known by those of skill in the art. “Exposure” to a factor, as used herein, means addition of factor(s) to the culture media and/or transfection of cells with constructs expressing the desired factor(s) and/or transfection of cells with constructs expressing transcription factors that permit the activation and deactivation of a differentiation factor or factors that cause the cell to differentiate.

In the present invention, cells are differentiated to form a desired cell type or types. The cells may be at least partly differentiated in the tissue formation reactor of the present invention. In this regard, the cells may be initially induced to differentiate in the cell growth and expansion reactor of the present invention, if desired. Once a desired percentage of the cells have differentiated (e.g., 50%, 60%, 70%, 80%, 90%, 95%, 98%, 99%, 100% or any percentage of cells between the percentages listed here) the cells are grown to confluency to form tissue. Confluency, as used herein, is defined, supra. In another embodiment, the cells are differentiated in the cell growth and expansion reactor and then transferred to the tissue formation reactor. This procedure may be best suited for non-attachment dependent cells. In yet another embodiment, a portion of the cells are differentiated in the cell growth and expansion reactor and a portion of the cells are differentiated in the cell differentiation and tissue formation reactor. In this embodiment, the percent of cells differentiated in the cell growth and expansion reactor may be 0%, 5%, 10%, 20%, 30%, 40%, 50%, 60%, 70%, 80%, 90%, 95%, 100% or any percentage of cells between the percentages listed here.

“Tissue” is defined herein, as an ensemble of predominately similar cells (and, sometimes, their extracellular matrix) from the same or similar origin that together carry out a specific function. Although tissues typically are made of predominately similar cells (e.g., muscle is predominately made of myocytes formed into myofibrils), other cell types may be included. For example, muscle tissue, in addition to myocytes, frequently also comprises adipocytes, fibroblasts, neurocytes, etc.

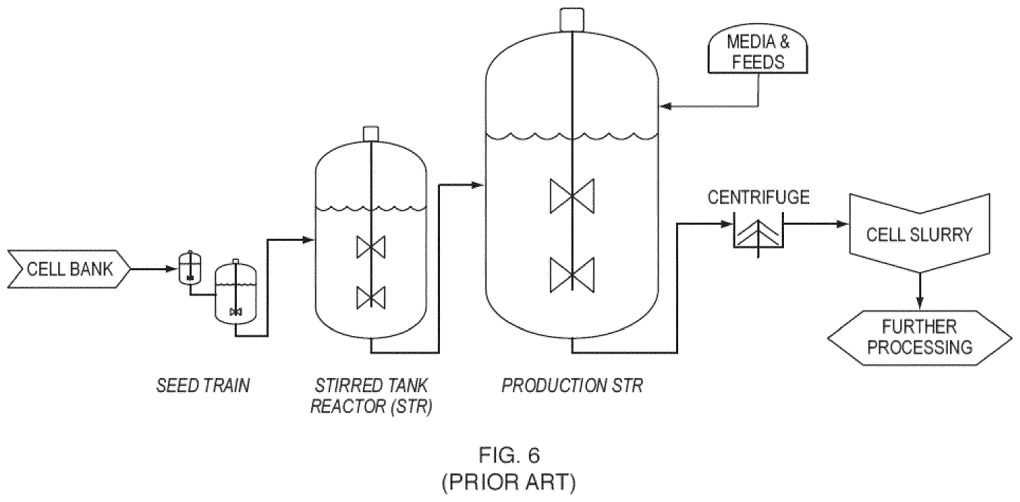

The process systems of the present invention may be used for the efficient and economical production of a structured clean meat product. The systems of the prior art (see, for example FIG. 6) are unable to efficiently or economically produce a structured meat product sufficiently meeting any of the criteria presented, below.

“Clean meat” is defined in the art as meat or a meat-like product (referred to collectively herein as “clean meat” or “clean meat product”) grown from cells in a laboratory, factory or other production facility suitable for the large-scale culture of cells.

A “structured meat product” or “structured clean meat product” is a meat product or clean meat product having a texture and structure like, similar to or suggestive of natural meat from animals. The structured meat product of the present invention has a texture and structure that resembles natural meat 1) in texture and appearance, 2) in handleability when being prepared for cooking and consumption (e.g., when being sliced, ground, cooked, etc.) and 3) in mouth feel when consumed by a person.

A “closed environment” as defined herein, refers to a system or culture system that does not expose the cells, culture medium or culture atmosphere to the external atmosphere. In comparison, an open culture system is exemplified by Petri dishes, culture flasks or microtiter plates. These expose the internal contents to the external atmosphere because gas exchange takes place by diffusion from under the lid or cap of the culture vessel. Sterility of these culture system relies on controlling the air flow around the vessel so that particles and other contaminants are not forced through the labyrinth that the gases must flow for proper gas exchange. Media is usually exchanged manually either on the bench top (sometimes in a stationary hood to block air flow) or in a sterile filtered laminar flow hood. In contrast, in a closed environment, any gas exchange takes place though filtered ports and media exchange is from/to sterilely and fluidly connected feed vessels and waste vessels.

The present invention also contemplates that the process system is semi-continuous or continuous process. The term “continuous process,” as used herein, refers to a process for growing and differentiating cells, which includes two or more process steps (or unit operations), such that the output from one process step flows directly into the next process step in the process, without interruption and/or without the need to collect the entire volume of the output from a process step before performing the next process step. In a preferred embodiment, two or more process steps can be performed concurrently for at least a portion of their duration. In other words, in case of a continuous process, as described herein, it is not necessary to complete a process step before the next process step is started, but a portion of the sample is always moving through the process steps. The term “continuous process” also applies to steps within a process operation, in which case, during the performance of a process operation including multiple steps, the sample flows continuously through the multiple steps that are necessary to perform the process operation. One example of such a process operation described herein is the flow through cell culture operation which includes multiple steps that are performed in a continuous manner and employs at least one cell growth and expansion reactor, one or more of cell differentiation and tissue formation reactors and, optionally, one or more cell retention devices.

Continuous processes, as described herein, also include processes where the input of the fluid material in any single process step or the output is discontinuous or intermittent. Such processes may also be referred to as “semi-continuous” or “fed-batch” processes. For example, in some embodiments according to the present invention, the input in a process step (e.g., cell seeding or media transfer) may be loaded continuously or semi-continuously. Further, the output i.e., harvesting) may be performed intermittently. Accordingly, in some embodiments, the processes and systems described herein include at least one-unit operation which is operated in an semi-continuous or intermittent matter, whereas the other unit operations in the process or system may be operated in a continuous manner.

The term “connected process” refers to a process for growing and differentiating cells, where the process comprises two or more process steps (or unit operations), which are connected to be in direct fluid communication with each other, such that fluid material continuously flows or semi-continuously flows through the process steps in the process and is in simultaneous contact with two or more process steps during the normal operation of the process. It is understood that at times, at least one process step in the process may be temporarily isolated from the other process steps by a barrier such as a valve in the closed position. This temporary isolation of individual process steps may be necessary, for example, during start up or shut down of the process or during removal/replacement of individual unit operations. The term “connected process” also applies to steps within a process operation which are connected to be in fluid communication with each other, e.g., when a process operation requires several steps to be performed in order to achieve the intended result of the operation (e.g., the cell growth, expansion and differentiation processes used in the methods described herein).

The present invention is not limited by the size of the cell growth and expansion reactor. Any available size reactor may be used in the present invention when used in accordance with the teachings of this specification. In one embodiment, the cell growth and expansion reactor is from 0.1 to 20,000 liters, 0.1 to 10,000 liters, 0.5 to 5,000 liters, 0.5 to 2,000 liters 0.5 to 1,000 liters, 0.5 to 800 liters, 0.5 to 500 liters, 0.5 to 300 liters, 0.5 to 100 liters and 0.5 to 20 liters. Further, the cell growth and expansion reactor may be any size that falls within any of the ranges given above, inclusive.

Further, the present invention is not limited by either the number or size of the tissue formation reactor(s). The size of the tissue formation reactors may be dependent upon, for example, the desired size of the tissue being produced, the physical limitations necessitate by the growth of the cells to confluency, the availability of reactors, etc. Likewise, the present invention is not limited to any particular number of tissue formation reactors. In one embodiment, the invention contemplates 1, 2, 3, 4, 5, 10, 25, 50, 75, 100 or more reactors in one process system, or any number of reactors in between the numbers specifically listed, as desired by one of skill in the art. The multiple tissue formation reactors may be seeded with cells from the cell growth and expansion reactor simultaneously, in parallel or in series (i.e., overflow from one cell differentiation and tissue formation reactor feeding the next). Likewise, the tissue formation reactors may be harvested simultaneously, or in series. When operated (seeded and harvested at confluency) in series, the cell growth and expansion reactor continuously supplies cells to newly installed tissue formation reactors as they are incorporated into the system, either as new positions or as replacement reactors for reactors that have been harvested. In this scenario, the cell growth and expansion reactor is not converted to a receptacle for differentiation media. The cell differentiation and tissue formation reactors may receive media from the cell growth and expansion reactors, for example, after passing the cells and media through the cell retaining devices, and returning a portion of the cells and a portion of the media to the cell growth and expansion reactor and a portion of the cells and the media to the tissue formation reactor(s). In this case, the cells in the cell growth and expansion reactor and the tissue formation reactor utilize the same media. In another scenario, additional constituents may be added to the media after the media has been separated in the cell retention device and prior to being feed into the tissue formation reactor to supplement the media coming from the cell growth and expansion reactor. In yet another scenario, additional constituents may be added directly to the tissue formation reactor to supplement the media coming from the cell growth and expansion reactor. In still yet another scenario, a separate vessel may be used to supply differentiation and/or growth media to the tissue formation reactors. Differentiation media is cell culture media used to induce stem cells (e.g., iPSCs) to differentiate into a desired cell type or cells having characteristics of a desired cell type.

If more than one tissue formation reactors are used, the system may utilize a manifold system for directing media and other ingredients to the reactors, as desired. Further, the manifold system can be used to isolate any one or more reactors for harvesting and replacement (or other manipulation) and maintain the integrity (e.g., sterility) of the remaining system components. The manifold system may be operated manually or be automated or semi-automated. Control systems, including computer control systems, that automate the manifold or other parts of the process system are also embodied by the present invention and discussed in greater detail, infra.

The process system of the present invention may also comprise monitoring systems for monitoring and analyzing the culture conditions and the media. The monitoring systems may comprise one or more systems (including sensors and probes) for measuring i) dissolved oxygen, ii) pH, iii) carbon dioxide, iv) cell waste, v) one or more cell metabolites, vi) temperature, vii) flow rate, viii) cell density and ix) cell viability. Suitable sensors and probes are known to one of skill in the art. The reactor conditions may be monitored in the cell growth and expansion reactor, in a sampling chamber fluidly connected with the cell growth and expansion reactor, in one or more of the tissue expansion reactors, in a sampling chamber fluidly connected to one or more of the tissue formation reactors, or in any other part of the system where one of skill in the art would understand that samples representative of the culture conditions in the system may be obtained.

In some embodiments, sensors and/or probes may be connected to a sensor electronics module, the output of which can be sent to a terminal board and/or a relay box. The results of the sensing operations may be input into a computer-implemented control system (e.g., a computer) for calculation and control of various parameters (e.g., temperature, pH, dissolved gases) and for display and user interface. Such a control system may also include a combination of electronic, mechanical, and/or pneumatic systems to control process parameters. It should be appreciated that the control system may perform other functions and the invention is not limited to having any particular function or set of functions.

In some embodiments of the present invention, a cell retention device may be utilized for separating cells from media. This may be desired, for example, when cells are transferred from the cell growth and expansion reactor to the tissue formation reactor. A cell retention device need not be required in each and every embodiment of the present invention or used during all steps in a process cycle. For example, in some embodiments, the cell retention device may be present but bypassed. In other embodiments, the cell retention device may be eliminated entirely. Processes of the present invention where the cell retention device is bypassed or eliminated, the function of the cell retention device, i.e., separation of cells and media, may be performed, for example, by either the cell growth and expansion reactor and/or the tissue growth reactor. For example, when cells from the cell growth and expansion reactor are seeded into the tissue growth reactor, the cells will be retained by the tissue growth reactor and the media can be directed into, for example, a waste vesicle.

The tissue formation reactor(s) of the present invention may be any device suitable for the differentiation and/or growth of cells into the desired tissue. Suitable reactors known in the art include, but are not limited to, hollow fiber reactors and reactors comprising other types of scaffolding known to one of skill in the art suitable for cell attachment and growth.

The process system of the present invention is not directed toward the culture of any particular cell type. Preferably, undifferentiated or de-differentiated cells are utilized and are differentiated in the system. However, the process system of the present invention may also be utilized for the culture of differentiated cells.

Cell culture parameters will be determined by the cell types to be cultured. Cell culture parameters include, but are not limited to, media, additional media components, media exchange rate, temperature, pH, gas exchange rate, etc. Further, the cell culture parameters may change as the cells differentiate and grow. For example, during differentiation specific growth factors may be required. During expansion, higher volumes of media exchange and/or gas exchange may be needed. One of skill in the art will be able, with the guidance of this specification, to determine the cell culture parameters for the cell type being cultured.

Unless defined otherwise, all technical and scientific terms used herein have the same meaning as commonly understood by one of ordinary skill in the art to which this invention pertains.

As used herein, the singular forms “a,” “an,” and “the” include plural unless the context clearly dictates otherwise.

As used herein, the transitional phrases “comprising,” “consisting essentially of” and “consisting of” have meanings as given in MPEP 2111.03. Any claim using the transitional phrase “consisting essentially of” will be understood to recite only essential elements of the invention. Any claim dependent from a claim reciting “consisting essentially of” will be understood to recite elements that are not essential to the invention.

All ranges include all values within the cited range including all whole, fractional and decimal numbers, inclusive.

This invention is further illustrated by the following examples which should not be construed as limiting. The contents of all references, patents and published patent applications cited throughout this application, as well as the Figures, are incorporated herein by reference.

### Exemplification

### Example 1

The process system of the present invention may be run in batch mode, fed batch mode and continuous mode. This example describes running the process system of the present invention in batch mode. The process system of the present invention may be used, for example, to produce a structured meat product. That process is exemplified here.

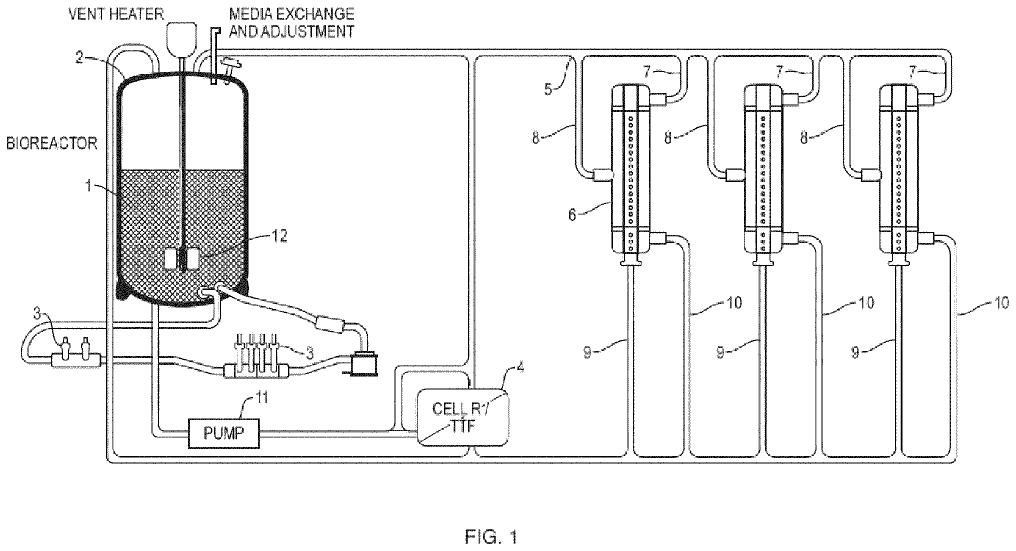

The process system is set up and connected essentially as displayed in FIG. 1. The process system comprises at least a cell growth and expansion reactor 2, a tissue growth reactor(s) 6 and, optionally, a cell retention device 4. Other set ups can be envisioned and utilized to one of skill in the art in view of the teachings of this specification and are included herein.

Proper installation of the growth and expansion reactor, cell retention device and tissue formation reactor are completed including making the necessary sterile connections. One an embodiment, there can be more than one growth and expansion reactor. A disposable reactor bag is used in the one or more growth and expansion reactors. Sensors (for example, 3) are connected and culture control parameters are established and entered into a control device (e.g., a computer).

The batch mode consists of filling the reactor with medium and inoculum (seed cell suspension at approximately 1×10⁶ cells/ml) and operating at predetermined parameters and adjusting the bioreactor and/or medium as need or indicated through the sensors including pH (approximately 6.8-7.3), carbon dioxide (approximately 5%), oxygen, temperature (approximately 37° C.), etc.

The medium used for this step is defined for cell proliferation and, as such, may be serum-based medium, serum-free or xeno-free medium. Xeno-free medium is defined herein as meaning a formulation only comprised of components derived from a single organism (e.g., bovine, porcine, etc.) and does not incorporate components from a foreign species. Xeno-free medium may or may not be serum-free. The components may be naturally derived or engineered. One of skill in the art can select a suitable medium for the cultured cell type with the guidance of this specification.

The cells used to seed the bioreactor in this example are iPSC but may be any desired cell. The cells may be suspension cells or adherent cells. For adherent cells, it is desirable for a screen or other device to be used at the outlet of the bioreactor in order to limit the size of cell aggregates. This helps to create a homogeneous culture in the bioreactor. The screen is used to calibrate the aggregates and limit their size allowing a good flow of media and therefore nutrients to the cells (if the aggregates are too big the cells on the inside of the aggregates will not survive as they will not get any nutrients from the media). The screen could be positioned at the outlet of the bioreactor before the retention system or also just after the retention device on the recirculation loop into the bioreactor.

The cells may be differentiated either in the growth and expansion reactor 2 or the tissue formation reactor(s) 6. This is dependent at least partially on the cell type(s) being cultured. For example, it is preferred to differentiate cells that are adherent after loading into the tissue formation reactor to avoid the step of detaching the cells from surfaces in the cell growth and expansion reactor.

If the cells are to be differentiated in the cell growth and expansion reactor, once the growth profile of the cell has been reached, the next step is to exchange the medium with a dedicated medium for the differentiation process, using the recirculation circuit via the cell retention device and system. As with the cell growth phase of the culture, the medium may be serum-based medium or serum-free or xeno-free medium. The medium may be the same as is used for the growth of the cells or may be a dedicated medium to induce the differentiation of the cells to the desired cell type. The cell type(s) desired in this example are one or more of bovine myocytes, bovine myocyte-like cells or cells engineered to have characteristics of bovine myocytes.

In an alternative procedure, the cells are transferred to the tissue formation reactor prior to differentiation. As discussed above, this is the preferred method for cells that are attachment dependent upon differentiation.

After the growth and, if desired, the differentiation step, the cells and the medium will be seeded into a tissue formation (and differentiation) reactor, for example, a hollow fiber device. The cell density in the cell growth and expansion reactor is approximately 1×10⁹ to 1×10¹² cells/ml. The cells are transferred via the cell retention device. The cell retention device separates the cells from the spent medium and, optionally, filters out cell aggregates. In batch mode, the transfer continues until the total transfer of the biomass from the bioreactor is completed. The bioreactor than is then used as a medium container and will continue to feed the cells in the tissue formation reactor until harvest. The cells will be fed with a medium suitable for growth (and, if necessary, differentiation) until the cells in the tissue formation reactor grow into the desired cell type (for example, myocytes or myocyte-like cells) and final desired level of confluency and tissue structure (for example, myofibrils giving a look and texture resembling natural meat), at which time they will be harvested. Spent media can be removed from the system after exiting from the tissue formation reactors and replaced partially or completely by fresh media.

Upon harvesting, further processing of the structured cultured meat product is performed if desired including adding flavorings, fats and further texturing.

In this example the final product is a cultured meat product having a look, texture, handleability and taste resembling natural meat. However, one of skill in the art will be able to create other desired products with the process system of the present invention in view of the teachings of this specification.

### Example 2

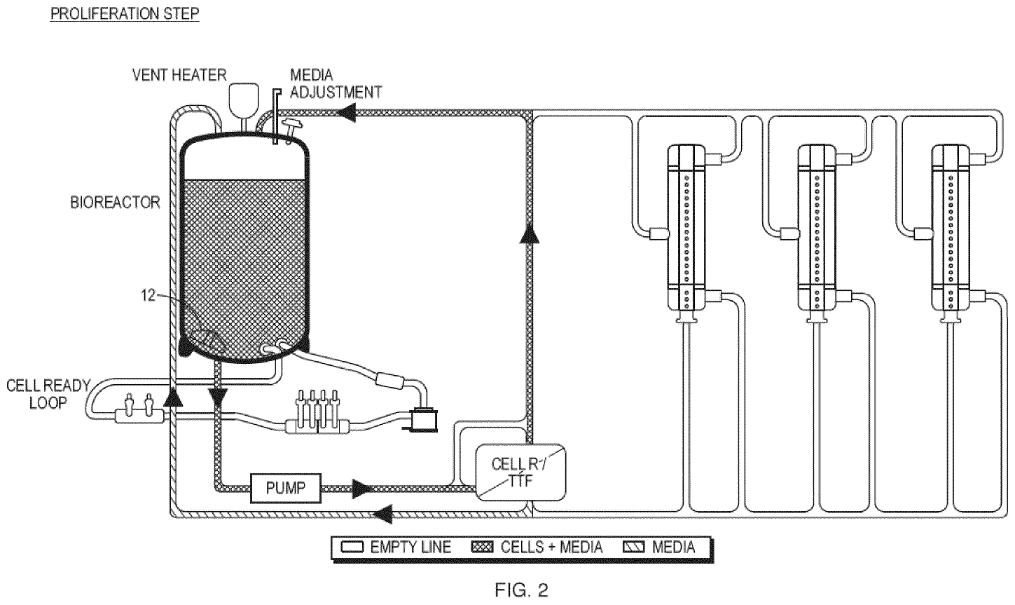

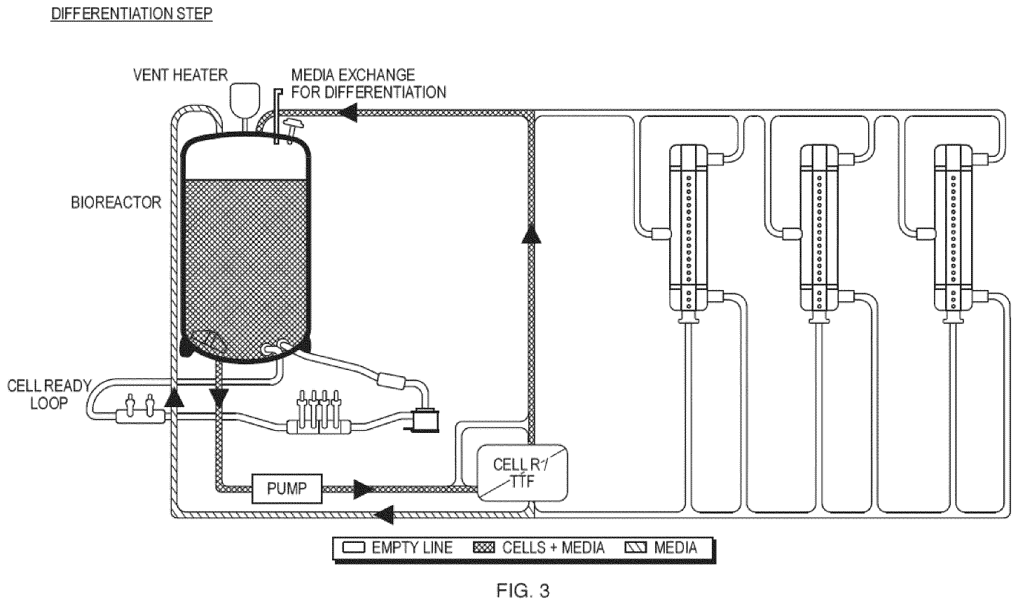

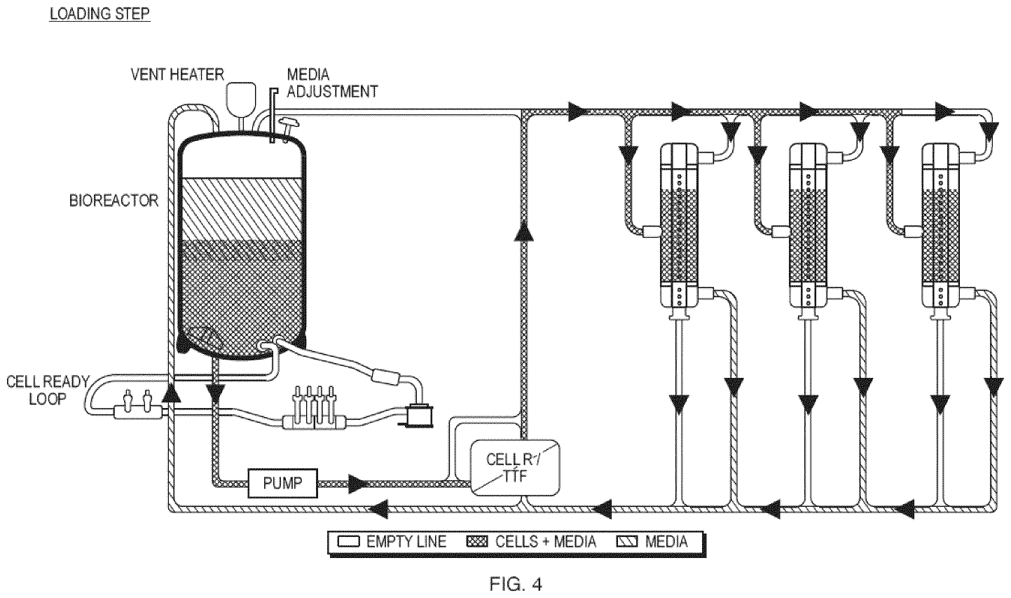

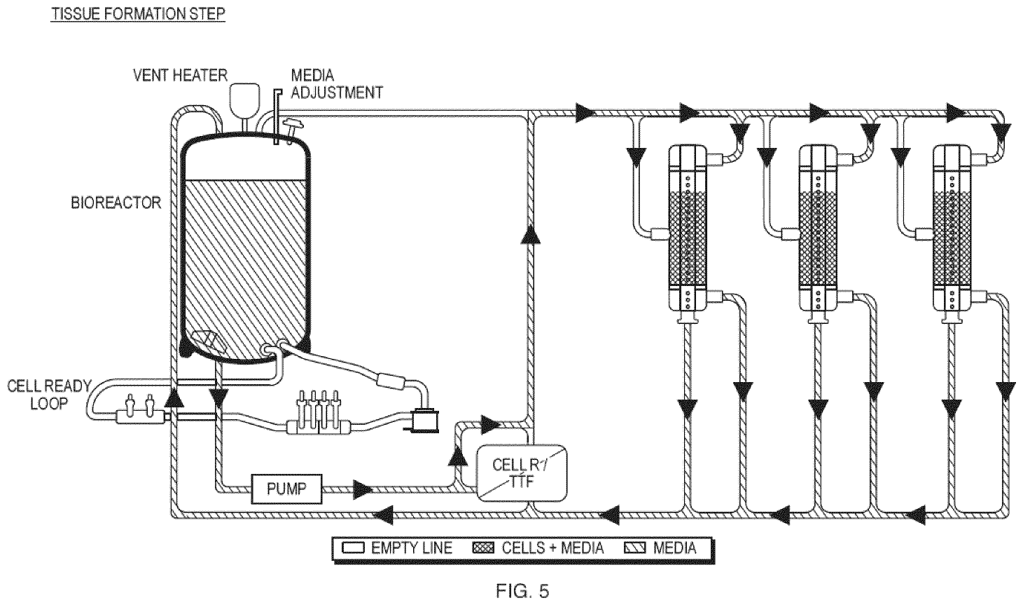

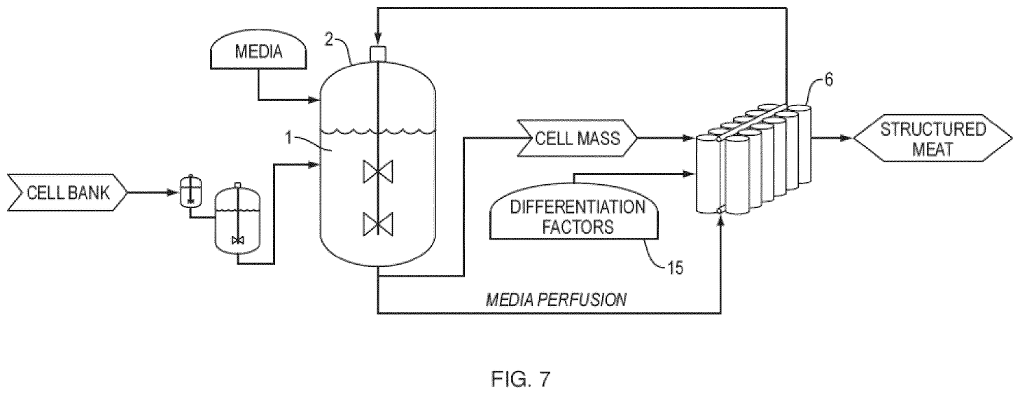

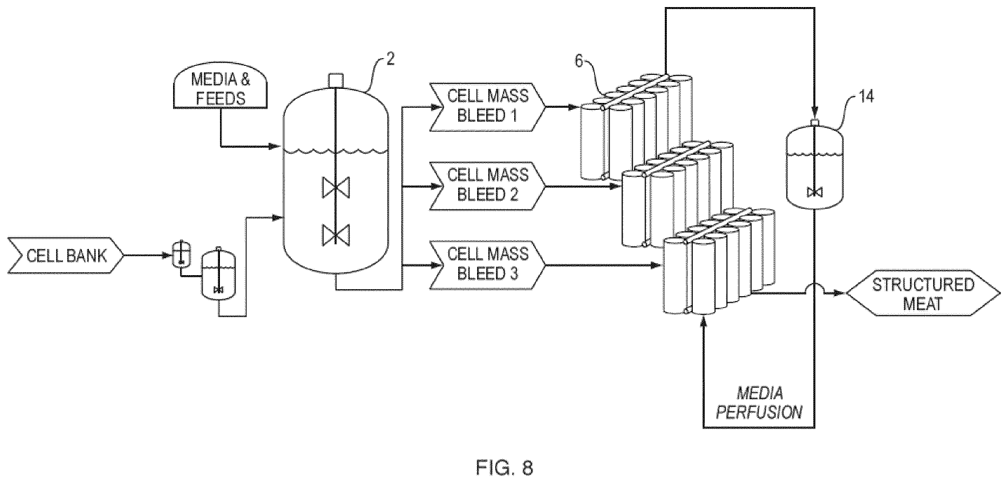

The process system of the present invention is also be performed in fed-batch and continuous mode. In fed-batch (semi-batch) mode, cells grown and expanded in the cell growth and expansion reactor are intermittently delivered to one or more tissue formation reactors (FIGS. 1 & 8). In this process system the growth and expansion reactor is not converted to a medium container. Rather, a separate vessel (see, FIG. 8; 14) is used as a medium container for feeding the tissue formation reactor(s). Cell transfer is intermittently interrupted to allow for further cell growth and expansion or reseeding, as necessary. Also, in this mode, the tissue formation reactors are harvested in series as each one reaches confluency and replaced with new reactors. FIGS. 2-5 show the various steps in this aspect of the present invention. Cell proliferation (FIG. 2) circulates media through process probes (no. 3 in FIG. 4) to monitor the culture condition and cell growth. Media is exchange for differentiation media (FIG. 3) and cells are allowed to differentiate in the bioreactor. Upon obtaining the correct cell density of differentiated cells, the cells are transferred to the tissue formation reactors after, optionally, being processed through the cell retaining device. See, FIG. 4. This may be referred to as the loading step. FIG. 5 shows the tissue formation step where cells are grown in the tissue formation reactors to desired confluency. FIG. 7 shows differentiation factors being added to the tissue formation reactors from a separate vessel 15 for embodiments where differentiation at least partially takes place in the tissue formation reactors. FIG. 8 shows three banks of tissue formation reactors. These banks of reactors may be seeded at different times and, thus, harvested and reseeded at different times, thereby making the process a continuous process. Spent media can be removed from the system after exiting from the tissue formation reactors and replaced completely or partially by fresh media.

Continuous mode resembles fed-batch mode however cell growth and expansion is at a rate that permits continuous transfer of cells to the tissue formation reactors. In this mode more than one cell growth and expansion reactors may be used.

We claim:

1. A closed environment process for the culture of cells to confluency to form tissue, said process comprising:
a) providing, a system comprising: i) a cell growth and expansion reactor; ii) one or more tissue formation reactors; and, iii) a cell retention device;
b) i) seeding said cell growth and expansion reactor and expanding the cell density within the cell growth and expansion reactor to a desired cell density, ii) once a desired cell density is obtained, processing the cells through the cell retention device thereby transferring the cells to the one or more tissue formation reactors and removing the growth media; and, iii) converting the bioreactor to a differentiation media reservoir for feeding the tissue growing reactors, and;
c) i) differentiating and growing said cells in said one or more tissue formation reactors until a desired level of confluency is reached and tissue formed, and ii) harvesting the tissue from said one or more tissue formation reactors.

2. The process of claim 1, wherein said process system is semi-continuous or continuous.

3. The process of claim 1, wherein the size of said cell growth and expansion reactor is from 0.5 liter to 20,000 liters.

4. The process of claim 3, wherein said the size of said cell growth and expansion reactor is from 0.5 liters to 2000 liters.

5. The process of claim 1, further comprising a manifold system to integrate said tissue formation reactors if said process system has two or more of said tissue formation reactors.

6. The process of claim 1, further comprising one or more monitoring systems for i) dissolved oxygen, ii) pH, iii) carbon dioxide, iv) cell waste, v) one or more cell metabolites, vi) temperature, vii) flow rate, viii) cell density and ix) cell viability.

7. The process of claim 1, further comprising, wherein the cell retention device can be bypassed.

8. The process of claim 1, wherein said one or more tissue formation reactors are hollow fiber reactors.

9. The process of claim 1, wherein, tissue can be sterilely harvested from one or more of the one or more tissue formation reactors while maintaining the sterility of the remainder of the system.

10. The process of claim 1, wherein the cells in the bioreactor are adapted to suspension growth, aggregate growth or microcarrier growth.

11. The process of claim 1, wherein said one or more tissue formation reactors comprise scaffolding for cell attachment.

12. A closed environment process for the culture of cells to confluency to form tissue, said process comprising:
a) providing: i) a cell growth and expansion reactor, ii) one or more tissue formation reactors; iii) a cell retention device;
b) i) seeding said cell growth and expansion reactor and expanding the cell density within the cell growth and expansion reactor, ii) once a desired cell density is obtained, iii) processing the cells through the cell retention device thereby transferring a portion of the cells to the one or more tissue formation reactors and returning a portion of the cells to the bioreactor; and, iv) continuing to transfer cells from the cell growth and expansion reactor to the one or more tissue formation reactors as adequate cell density become available in the cell growth and expansion reactor; and,
c) i) differentiating and growing said cells in said one or more tissue formation reactors until a desired level of confluency is reached and tissue formed, and ii) harvesting the tissue from said one or more tissue formation reactors.

13. The process of claim 12, further comprising a first reservoir holding cell growth media and a second reservoir holding differentiation media, said cell growth media being delivered to the cell growth and expansion reactor and said differentiation media being delivered to the one or more tissue formation reactors after transferring said cells to the one or more tissue formation reactors.

12. he process of claim 12, wherein said process system is semi-continuous or continuous.

15. The process of claim 12, wherein the size of said cell growth and expansion reactor is from 0.5 liters to 20,000 liters.

16. The process of claim 15, wherein the size of said cell growth and expansion reactor is from 0.5 liter to 2000 liters.

17. The process of claim 12, further comprising a manifold system to integrate said tissue formation reactors if said process system has two or more of said tissue formation reactors.

18. The process of claim 12, further comprising one or more monitoring systems for i) dissolved oxygen, ii) pH, iii) carbon dioxide, iv) cell waste, v) one or more cell metabolites, vi) temperature, vii) flow rate, viii) cell density and ix) cell viability.

19. The process of claim 12, further comprising, wherein the cell retention device can be bypassed.

20. The process of claim 12, wherein said one or more tissue formation reactors are hollow fiber reactors.

21. The process of claim 12, wherein, tissue can be sterilely harvested from one or more of the one or more tissue formation reactors while maintaining the sterility of the remainder of the system.

22. The process of claim 12, wherein the cells in the bioreactor are adapted to suspension growth, aggregate growth or microcarrier growth.

23. The process of claim 12, wherein said one or more tissue formation reactors comprise scaffolding for cell attachment.

24. The process of claim 12, further comprising a separate reservoir for differentiation media that is fluidly connected to said tissue formation reactors.
